(19) United States
(12) Patent Application Publication
(10) Pub. No.: US 2007/0041930 A1
(43) Pub. Date: Feb. 22, 2007

(54) Use of quaternary polysiloxanes in cleaning and care compositions

(75) Inventors: Markus Meder, Kelkheim (DE); Sabine Haala, Hanau (DE); Carsten Mueller, Frankfurt am Main (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant Produkte (Deutschland) GmbH)

(21) Appl. No.: 11/506,019

(22) Filed: Aug. 17, 2006

(30) Foreign Application Priority Data
Aug. 20, 2005 (DE) 102005039511.2

(51) Int. Cl. A61K 8/89

(52) U.S. Cl. 424/70.122

(57) ABSTRACT

The invention relates to the use of polysiloxanes which comprise a plurality of quaternary ammonium groups in the molecule in cosmetic or pharmaceutical compositions for the cleaning and care of keratin fibers, in particular of human hair. The improvement in the shine and in the color protection of the hair is achieved if the polysiloxanes are used in an amount in the range from 0.01 to 30%.

The invention relates to the use of polysiloxane compounds containing quaternary ammonium groups for the cleaning and care of keratin-containing substrates, in particular of human hair.

It is known that polysiloxane-based quaternary compounds (quats) can be used advantageously in care compositions for keratin-containing substrates, in particular for haircare compositions. EP-A 282 720 and DE-A 37 19 086 claim quaternary polysiloxanes and their use in conditioning haircare products. Although these quaternary polysiloxanes mostly have a softening effect, improve the combability and reduce the electrostatic charging of the hair, they are inadequate with regard to the color stabilization of colored hair.

WO 2005/035 628 describes polyquaternary polysiloxanes and their use as softening component in the textile industry in the manufacture of textiles.

It was the object to provide substances for the treatment of keratin fibers which bring about good conditioning, e.g. of skin, hair and textiles, improve the color brilliance and the shine, can easily be incorporated into formulations, produce as clear an appearance as possible and which exhibit a conditioning effect. Moreover, the compositions should have good “substantivity”, i.e. for tinted or colored hair, bring about an improvement in the color absorption behavior and an increase in the color stability and shape retention. In particular, there is a need for haircare and cleaning compositions which prevent the color being washed out of tinted and colored hair and protect the hair upon exposure to heat and sun.

Surprisingly, it has been found that this object is achieved through polysiloxanes which comprise a plurality of quaternary ammonium groups.

The invention thus provides agents for the cleaning and care of keratin fibers, in particular of human hair, comprising polyquaternary polysiloxanes of the formula (S1) 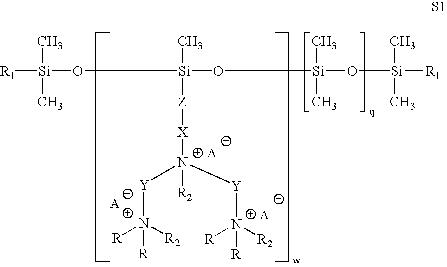
in which

the sum of (q+w) has a range from 10 to 1500, preferably from 15 to 600, and the ratio q/w has a range from 5 to 600, preferably from 10 to 400,

R is C1-C4-alkyl, linear or branched,

R1 is hydrogen, C1-C3-alkyl or C1-C3-alkoxy,

R2 is C1-C7-alkyl or benzyl,

X is a direct bond, 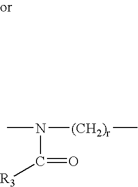
in which

r is an integer from 1 to 4, and

R3 is C1-C7-alkyl or —NH—C1-C7-alkyl, 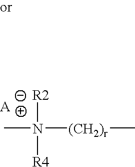
in which

R2 and r are as defined previously and

R4 is C1-C3-alkyl, 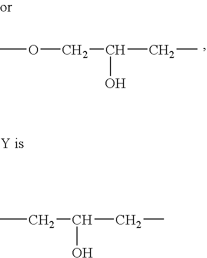
or

—(CH2)x-,
in which x is an integer from 1 to 4,

Z is C2-C4-alkylene, linear or branched,

A- is CH3OSO3-, chloride, bromide, iodide or tosylsulfate-
or of the formula (S2) 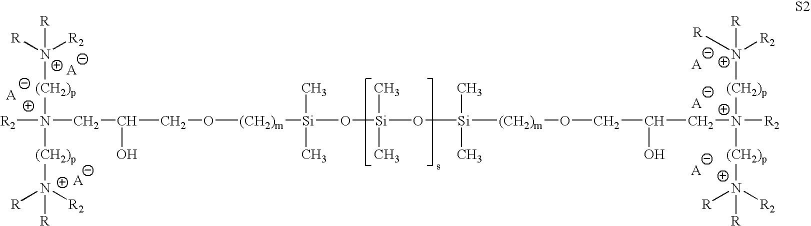
in which

R, R2 and A- have the same meaning as in formula (S1),

m is an integer from 1 to 4,

p is an integer from 1 to 4, and

s is a number in the range from 5 to 1500, preferably from 10 to 600.

Preference is given to compounds in which
R is methyl, ethyl, or propyl,
R1 is H, methyl, —OCH3 or —OC2H5,
R2 is methyl or benzyl,
R3 is methyl or —NH—C4H9,
R4 is methyl,
A- is CH3OSO3- or chloride,

Z is C3-alkylene, linear or branched,

m is 3,

p is 3

s is 10 to 600,

r is 2 and

x is 3.

Of very particular suitability are polysiloxanes with the following structural units:

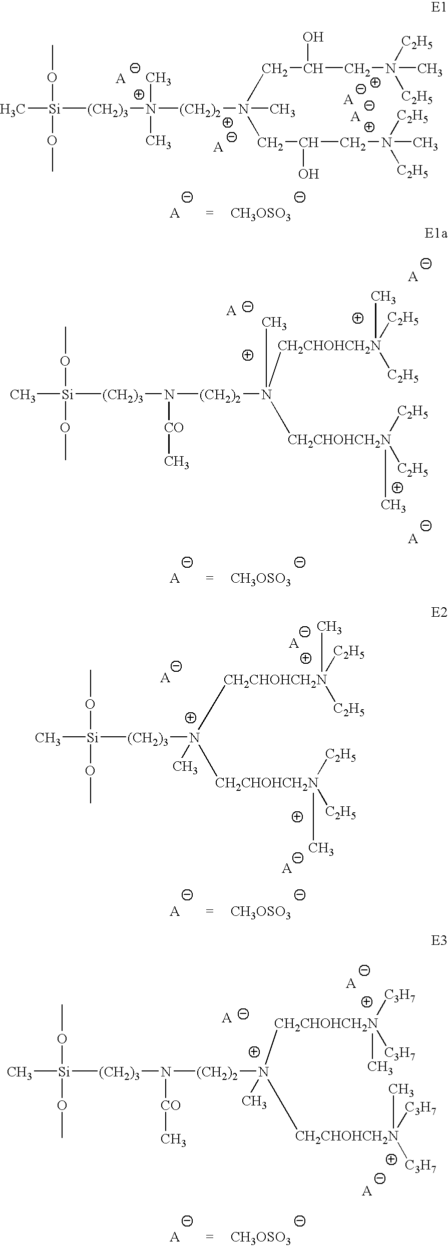

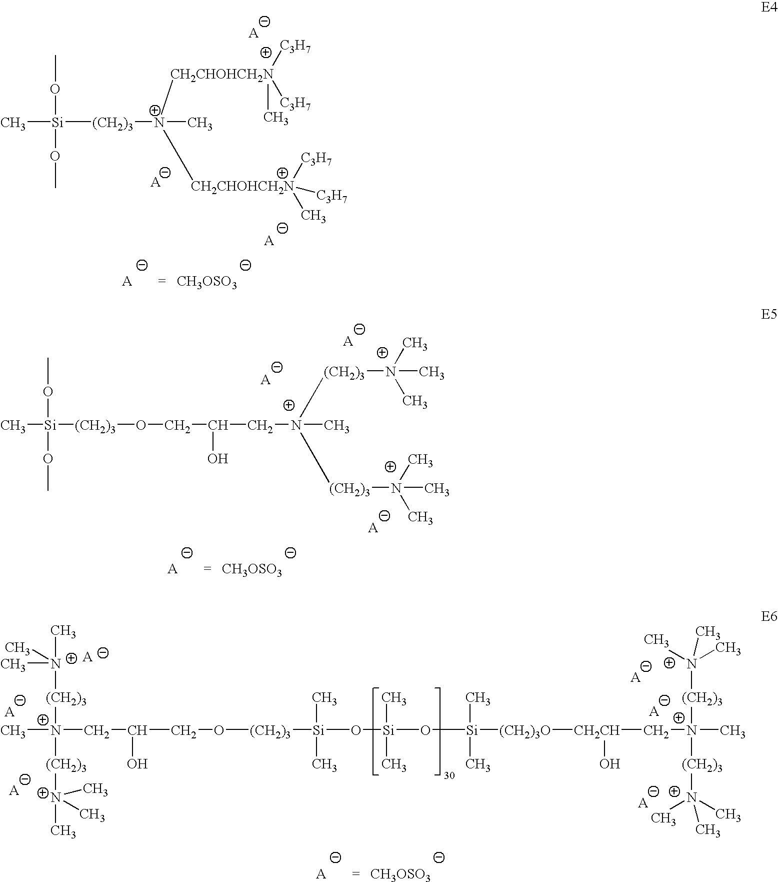

Polysiloxanes with the structural units E1 are extraordinarily preferred.

The abovementioned polysiloxanes are prepared in the following way:

The compounds of the formula (S1) in which Y is 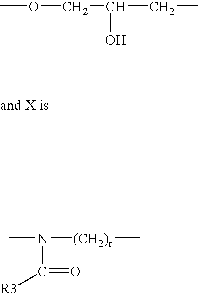
can be prepared by reacting 3-(2-aminoalkylamino)alkyldialkoxymethylsilane with glycidyidialkylamine to the corresponding silane, and subsequently reacting the formed silanes with a) polydimethylsiloxanediol or with octamethylcyclotetrasiloxane, and with b) tetraalkyl- or arylalkylammonium hydroxide (e.g. benzyltrimethyl-, tetramethyl- or tetrabutylammonium hydroxide) to give polysiloxanes with subsequent quaternization to the polyquaternary siloxanes. Preferred starting substances are 3-(2-aminoethylamino)propyldimethoxymethylsilane, 3-(2-aminoethylamino)propyldiethoxymethylsilane and glycidyldimethylamine, glycidyidiethylamine and glycidyldipropylamine. Examples thereof are the end products E1a and E3.

For the quaternization, it is possible to use quaternizing agents known per se as are used for the quaternization of tertiary amino groups, e.g. alkyl halides or dialkylsulfates, e.g. dimethyl sulfate, diethyl sulfate or methyl or ethyl chloride or bromide, or benzyl chloride. Here, it is advantageous to use benzyl chloride or preferably a dialkyl sulfate for this. Here, the corresponding counterion (particularly chloride or alkyl sulfate ion) to the particular formed quaternary ammonium ion arises. Particular preference is given to dimethylsulfate.

The polysiloxanes of the formula (S1) in which Y is 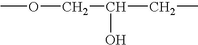
and X is a direct bond, can be prepared by reacting 3-aminoalkyl-dialkoxy-methylsilane with glycidyldialkylamine (preparable by reacting dialkylamine with epichlorohydrin) to give the corresponding silane, and subsequently reacting the formed silanes with a) polydimethylsiloxanediol or with octamethyl-cyclotetrasiloxane, and with b) tetraalkyl- or arylalkyl-ammonium hydroxide (e.g. benzyltrimethyl-, tetramethyl- or tetrabutylammonium hydroxide) to give polysiloxanes with subsequent quaternization to the polyquaternized siloxanes.

Preferred starting substances are 3-aminopropyl-diethoxy-methylsilane, 3-aminopropyldimethoxymethylsilane and glycidyldimethylamine, glycidyldiethylamine and glycidyldipropylamine. Examples thereof are the structural units E2 and E4.

The polysiloxanes of the formula (S1) in which Y is —(CH2)x- and X is 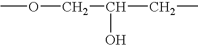
can be prepared by reacting N′-[3-(dialkylamino)alkyl]-N,N-dialkylalkane-1,3-diamine with dialkoxy(3-glycidyloxyalkyl)methylsilane and subsequent reaction with polydimethylsiloxanediol or with octamethylcyclotetrasiloxane with subsequent quaternization.

Preferred starting substances are N′-[3-(dimethylamino)propyl]-N,N-dimethylpropane-1,3-diamine, diethoxy-(3-glycidyloxypropyl)methylsilane and dimethoxy(3-glycidyloxypropyl)methylsilane. One example of this which may be mentioned is the structural unit E5.

The compounds of the formula (S2) can be prepared, for example, by reacting octamethylcyclotetrasiloxane with 1,1,3,3-tetraalkyldisiloxane, preferably 1,1,3,3-tetramethyldisiloxane, reacting the reaction product with an allyl glycidyl ether and a hydrosilylation catalyst, reacting this reaction product with N,N,N′,N′-tetraalkyldialkylenetriamine, preferably N,N,N′,N′-tetramethyldipropylenetriamine, to give the polysiloxane, and subsequent quaternization. One example of this which may be mentioned is the structure E6.

Instead of octamethylcyclotetrasiloxane, it is also possible to use penta- or hexamethylcyclotetrasiloxane or mixtures thereof.

The polyquaternary polysiloxanes used according to the invention exhibit excellent substantivity toward keratin fibers, and also good conditioning and color-retaining to color-intensifying effects, in particular toward hair. It is particularly advantageous that haircare and hair-cleaning compositions comprising polyquaternary polysiloxanes defined above prevent or minimize the colors being washed out of tinted and colored hair.

The color absorption behavior of hair colorants can be improved through the polyquaternary polysiloxanes used according to the invention. In addition, in hair styling compositions, a volumizing and shine-imparting effect of the polyquaternary polysiloxanes is significant. Further advantages are the good solubility in water, a favorable viscosity behavior, the good incorporability, and a clear appearance of the polyquaternary polysiloxanes used according to the invention. Moreover, they are insensitive toward UV and IR radiation. They are thus useful constituents of haircare and hair-cleaning compositions, and also hair colorants.

In one preferred embodiment of the invention, the cosmetic or pharmaceutical compositions are in aqueous, aqueous-alcoholic, alcoholic or aqueous-surface-active form, or they are compositions based on oil, or they are in emulsion, suspension or dispersion form, more specifically in the form of fluids, foams, sprays, gels, mousse, lotions or creams.

The emulsions may either be water-in-oil emulsions or oil-in-water emulsions, microemulsions, nanoemulsions and multiple emulsions. The emulsions can be prepared in a known way, e.g. for example by cold, hot, hot/cold or PIT emulsification.

The polyquaternary polysiloxanes used according to the invention in cosmetic compositions have marked hydrophilicity which can be modified by incorporating appropriate groups and substituents.

Good substantivity, conditioning effect, and shine-imparting and volumizing effects of the polyquaternary polysiloxanes described above make them of use according to the invention for producing hair-treatment compositions, preferably shampoos, hair conditioners, hair treatments, styling compositions, hair rinses, volume spray, styling fluid, hair foam, hair gel, setting composition, hairspray, mousse, hair oils and end fluids.

The polyquaternary polysiloxanes described above improve the color absorption behavior of hair colorants and are thus useful constituents in hair tinting and coloring compositions. At the same time, they are additionally color-protection additives and improve the durability of hair tints or permanent hair colorants and significantly increase the shine, in particular of colored hair.

In addition, the invention thus also provides the use of a cosmetic or pharmaceutical composition according to the invention for protecting and for retaining the color in colored keratin fibers, preferably in colored human hair. Preferably, the composition according to the invention comprises for this use from 0.01 to 10% by weight, based on the finished composition, of quaternary polysiloxane.

In preferred embodiments, the haircare and cleaning compositions comprise UV filters.

In a further preferred embodiment of the invention, the cosmetic and pharmaceutical compositions are surfactant-free compositions, surfactant-free emulsions, gels, sprays, spray foams, mousse or fluids.

In a further preferred embodiment of the invention, the cosmetic or pharmaceutical compositions are additives for permanent waving compositions, in particular conditioners for the after-treatment.

The cosmetic or pharmaceutical compositions according to the invention on an aqueous or aqueous-alcoholic basis comprise polyquaternary polysiloxanes preferably in the amounts by weight of from 0.01 to 30%, particularly preferably from 0.2 to 10%, especially preferably from 0.5 to 2%, based on the finished compositions.

The cosmetic or pharmaceutical compositions according to the invention in the form of an emulsion comprise polyquaternary polysiloxanes preferably, in amounts by weight, from 0.01 to 30%, particularly preferably from 0.05 to 10% and especially preferably from 0.1 to 5%, based on the finished composition.

In a further preferred embodiment, the compositions according to the invention are oil-in-water emulsions with a water fraction of from 5 to 95% by weight, preferably 15 to 75% by weight, particularly preferably 25 to 85% by weight.

For the compositions according to the invention on an aqueous-alcoholic or alcoholic basis, all mono- or polyhydric alcohols are suitable. Preference is given to alcohols having 1 to 4 carbon atoms, such as ethanol, propanol, isopropanol, n-butanol, isobutanol, t-butanol or glycerol, and alkylene glycols, in particular propylene glycol, butylene glycol or hexylene glycol, and mixtures of said alcohols. Further preferred alcohols are polyethylene glycols with a relative molecular mass below 2000. In particular, a use of polyethylene glycol with a relative molecular mass between 200 and 600 and of polyethylene glycol with a relative molecular mass between 400 and 600 is preferred.

The compositions according to the invention can comprise the following oils: hydrocarbon oils with linear or branched, saturated or unsaturated C₇-C₄₀-carbon chains, for example dodecane, isododecane, cholesterol, hydrogenated polyisobutylenes, docosanes, hexadecane, isohexadecane, paraffins and isoparaffins, but also triglycerides of animal and vegetable origin, for example beef tallow, pig fat, goose grease, perhydrosqualene, lanolin, sunflower oil, corn oil, soybean oil, rice oil, jojoba oil, babusscu oil, pumpkin oil, grapeseed oil, sesame oil, walnut oil, apricot oil, macadamia oil, avocado oil, sweet almond oil, lady's smock oil, castor oil, olive oil, peanut oil, rapeseed oil and coconut oil and synthetic oils such as purcellin oil, linear and/or branched fatty alcohols and fatty acid esters, preferably Guerbet alcohols having 6 to 18, preferably 8 to 10, carbon atoms; esters of linear (C₆-C₁₃)-fatty acids with linear (C₆-C20)-fatty alcohols; esters of branched (C₆-C₁₃)-carboxylic acids with linear (C₆-C₂₀)-fatty alcohols, esters of linear (C₆-C₁₈)-fatty acids with branched alcohols, in particular 2-ethylhexanol; esters of linear and/or branched fatty acids with polyhydric alcohols (such as, for example, dimerdiol or trimerdiol) and/or Guerbet alcohols; alcohol esters of C₁-C₁₀-carboxylic acids or C₂-C₃₀-dicarboxylic acids, esters, such as dioctyl adipate, diisopropyl dimer dilinoleate; propylene glycols/dicaprylate or waxes, such as beeswax, paraffin wax or microwaxes, optionally in combination with hydrophilic waxes, such as, for example, cetylstearyl alcohol; fluorinated and perfluorinated oils; monoglycerides of C₁-C₃₀-carboxylic acids, diglycerides of C₁-C₃₀-carboxylic acids, triglycerides of C₁-C₃₀-carboxylic acids, for example triglycerides of caprylic/capric acids, ethylene glycol monoesters of C₁-C₃₀-carboxylic acids, ethylene glycol diesters of C1-C30-carboxylic acids, propylene glycol monoesters of C₁-C₃₀-carboxylic acids, propylene glycol diesters of C₁-C₃₀-carboxylic acids, and propoxylated and ethoxylated derivatives of the abovementioned classes of compound. The carboxylic acids can comprise linear or branched alkyl groups or aromatic groups. By way of example, mention may be made of diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenate, dioctyl maleate, dioctyl sebacate, cetyl octanoate, diisopropyl dilinoleate, caprylic/capryl triglyceride, PEG-6 caprylic/capryl triglyceride, PEG-8 caprylic/capryl triglyceride, cetyl ricinoleate, cholesterol hydroxystearate, cholesterol isostearate, C₁-C₃₀-monoesters and polyesters of glyceryl, for example glyceryl tribehenate, glyceryl stearate, glyceryl palmitate, glyceryl distearate, glyceryl dipalmitate, C₁-C₃₀-carboxylic acid monoesters and polyesters of sugars, for example glucose tetraoleate, glucose tetraesters of soybean oil fatty acid, mannose tetraesters of soybean oil fatty acid, galactose tetraesters of oleic acid, arabinose tetraesters of linoleic acid, xylose tetralinoleate, galactose pentaoleate, sorbitol tetraoleate, sorbitol hexaesters of unsaturated soybean oil fatty acid, xylitol pentaoleate, sucrose tetraoleate, sucrose pentaoleate, sucrose hexaoleate, sucrose heptaoleate, sucrose oleate.

The silicone oils available are preferably dimethylpolysiloxanes and cyclomethicones, polydialkylsiloxanes R³SiO(R²SiO)ₓSiR³, where R is methyl or ethyl, particularly preferably methyl, and x is a number from 2 to 500, for example the dimethicones available under the trade names VICASIL (General Electric Company), DOW CORNING 200, DOW CORNING 225, DOW CORNING 200 (Dow Corning Corporation), trimethylsiloxysilicates [(CH₂)₃SiO)1/2]x[SiO₂]y, where x is a number from 1 to 500 and y is a number from 1 to 500, dimethiconols R³SiO[R²SiO]xSiR²OH and HOR²SiO[R²SiO]xSiR²OH, where R is methyl or ethyl and x is a number up to 500, polyalkylarylsiloxanes, for example the polymethylphenolsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty acid-, alcohol-, polyether-, epoxy-, fluorine- and/or alkyl-modified silicone compounds, and polyether siloxane copolymers.

The cosmetic and pharmaceutical compositions according to the invention comprise the abovementioned silicone oils in the amounts by weight of from 0.5% to 15%, preferably 1% to 10%, particularly preferably 1.5% to 5%.

The hair colorants and tints according to the invention comprise preferably direct dyes and/or oxidation dye precursors in the customary pH ranges. Suitable direct dyes are preferably nitroaniline derivatives, such as 1-[(2-hydroxyethyl)amino]-2-nitrobenzene (Velsol® Yellow 2), 4-hydroxypropylamino-3-nitrophenol (Velsol Red BN), 3-nitro-p-hydroxyethylaminophenol (Velsol Red 54), 4-hydroxyethylamino-3-nitroaniline (Velsol Red 3), N,N′-bis(hydroxyethyl)-2-nitro-p-phenylenediamine (Velsol Violet BS), N,N′,N′-tris-(hydroxyethyl)-2-nitro-p-phenylenediamine (Velsol Blue 2), 4-(2′-hydroxyethyl)amino-3-nitrotoluene, 4-(2′-hydroxyethyl)amino-3-nitrobenzyl alcohol, 4-(2′-hydroxyethyl)amino-3-nitro-1-trifluoromethylbenzene, 4-(2′,3′-dihydroxypropyl)amino-3-nitrochlorobenzene, 4-(2′-hydroxyethyl)amino-3-nitrobromobenzene and 4-(2′,3′-dihydroxypropyl)amino-3-nitrobromobenzene, nitrobenzene derivatives, for example 2-amino-4-nitrophenol, picramic acid, 1-[(2′-hydroxyethyl)amino]-2-amino-4-nitrobenzene, 2-nitro-4-[(2′-hydroxyethyl)amino]aniline, 4-bis[(2′-hydroxyethyl)amino]-1-methylamino-2-nitrobenzene, 2,5-bis[(2′-hydroxyethyl)amino]nitrobenzene, 2-(2′-hydroxyethyl)amino-4,6-dinitrophenol, 1-amino-4-(2′,3′-dihydroxypropyl)amino-2-nitro-5-chlorobenzene, but also triphenylmethane dyes such as, for example, Basic Violet 1 (C.I. 42535), azo dyes, such as, for example, Acid Brown 4 (C.I. 14805), anthraquinone dyes, such as, for example, Disperse Blue 23 (C.I. 61545), Disperse Violet 4 (C.I.61105), 1,4,5,8-tetraaminoanthraquinone and 1,4-diaminoanthraquinone and further direct dyes.

Oxidation dye precursors which are available are preferably p-phenylenediamines and p-aminophenols and derivatives thereof, such as, for example, p-tolylenediamine, p-phenylenediamine, p-aminophenol, which are combined with so-called modifiers or couplers, such as, for example, m-phenylenediamine, resorcinol, m-aminophenol and derivatives thereof for the purpose of nuancing the coloration.

Suitable oxidizing agents for developing hair colorations are preferably hydrogen peroxide and its addition compounds.

The polyquaternary polysiloxanes used according to the invention are very readily compatible with pearlescence-imparting components. The hair treatment compositions according to the invention can thus advantageously comprise pearlescent-imparting compounds, for example fatty acid monoalkanolamides, fatty acid dialkanolamides, monoesters or diesters of alkylene glycol, in particular ethylene glycol and/or propylene glycol or oligomers thereof with higher fatty acids, e.g. palmitic acid, stearic acid or behenic acid or mixtures thereof, monoesters or diesters of alkylene glycols with fatty acids, fatty acids and metal salts thereof, monoesters or polyesters of glycerol with carboxylic acids and ketosulfones of various types, preferably ethylene glycol distearate and polyethylene glycol distearate with about 3 glycol units.

The hair treatment compositions according to the invention comprise preferably 0.1 to 15% by weight, particularly preferably 1 to 10% by weight, of pearlescent-imparting compounds.

Glitter effects and shine effects of the compositions according to the invention can be produced preferably by adding mica, colored polyacrylic esters and mica, mica-iron oxide, mica-titanium oxide and through pigments. Suitable pigments are metal oxides, for example iron oxides, titanium oxide, ultramarine blue, and pigments modified with cationic coating shells, as described in WO 00/12053.

As further auxiliaries and additives, the hair treatment compositions according to the invention can comprise surfactants, emulsifiers, cationic polymers, thickeners, film formers, antimicrobial active ingredients, antioxidants, pigments/micropigments, gelling agents, and further additives customary in cosmetics, such as, for example, superfatting agents, moisturizing agents, silicones, stabilizers, further conditioners, glycerol, preservatives, pearlizing agents, dyes, fragrance and perfume oils, solvents, hydrotropes, opacifiers, fatty alcohols, antidandruff agents, vitamins, Bisabolol®, Allantoin®, Phytantriol®, Panthenol®, AHA acids, plant extracts, for example aloe vera and proteins.

Anionic washing-active substances which may be mentioned are preferably: C₁₀-C₂₀-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfates, alkanesulfonates and hydroxyalkanesulfonates, olefinsulfonates, acyl esters of isethionates, α-sulfofatty acid esters, alkylbenzenesulfonates, alkylphenol glycol ether sulfonates, sulfosuccinates, sulfosuccinic monoesters and diesters, fatty alcohol ether phosphates, protein-fatty acid condensation products, alkyl monoglyceridesulfates and sulfonates, alkyl glyceride ether sulfonates, fatty acid methyltaurides, fatty acid sarcosinates, sulforicinoleates, amphoacetates or amphoglycinates, acylglutamates. These compounds and mixtures thereof are used in the form of their water-soluble or water-dispersible salts, for example the sodium, potassium, magnesium, ammonium, mono-, di- and triethanolammonium and analogous alkylammonium salts.

The weight fraction of the anionic surfactants is preferably 1 to 30% by weight, particularly preferably 5 to 25% by weight, especially preferably 10 to 22% by weight, based on the finished compositions.

Suitable cationic surfactants are, for example, quaternary ammonium salts, such as di(C₁₀-C₂₄-alkyl)dimethylammonium chloride or bromide, preferably di(C₁₂-C₁₈-alkyl)dimethylammoniumchloride or bromide; C₁₀-C₂₄-alkyldimethylethylammoniumchloride or bromide; C₁₀-C₂₄-alkyltrimethylammoniumchloride or bromide, preferably cetyltrimethylammonium chloride or bromide and C₂₀-C₂₂-alkyltrimethylammoniumchloride or bromide; C₁₀-C₂₄-alkyldimethylbenzylammoniumchloride or bromide, preferably C₁₂-C₁₈-alkyldimethylbenzylammoniumchloride; N-(C₁₀-C₁₈-alkyl)pyridiniumchloride or bromide, preferably N-(C₁₂-C₁₆-alkyl)pyridiniumchloride or bromide; N-(C₁₀-C¹⁸-alkyl)isoquinolinium chloride, bromide or monoalkylsulfate; N-(C₁₂-C₁₈-alkylpolyolaminoformylmethyl)pyridiniumchloride; N-(C₁₂-C₁₈-alkyl)-N-methylmorpholiniumchloride, bromide or monoalkylsulfate; N-(C₁₂-C₁₈-alkyl)-N-ethylmorpholiniumchloride, bromide or monoalkylsulfate; C₁₆-C₁₈-alkylpentaoxethylammoniumchloride; diisobutylphenoxyethoxyethyldimethylbenzylammonium chloride; salts of N,N-diethylaminoethylstearylamide and -oleylamide with hydrochloric acid, acetic acid, lactic acid, citric acid, phosphoric acid; N-acylaminoethyl-N,N-diethyl-N-methylammonium chloride, bromide or monoalkylsulfate and N-acylaminoethyl-N,N-diethyl-N-benzylammoniumchloride, bromide or monoalkylsulfate, where acyl is preferably stearyl or oleyl.

The weight fraction of the cationic surfactants is preferably 0.1 to 10% by weight, particularly preferably 0.2 to 7% by weight, especially particularly preferably 0.5 to 5% by weight, based on the finished composition.

Nonionic and amphoteric surfactants are very advantageous. Suitable nonionic surfactants which can be used as washing-active substances are preferably fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid amide polyethylene glycols; N-alkyl-, N-alkoxypolyhydroxyfatty acid amide, in particular fatty acid N-methylglucamides, sucrose esters; polyglycol ethers, alkyl polyglycosides, phosphoric esters (mono-, di- and triphosphoric esters ethoxylated and nonethoxylated).

The weight fraction of the nonionic surfactants in the compositions according to the invention (e.g. in the case of rinse-off products) is preferably in the range from 1 to 20% by weight, particularly preferably 2 to 10% by weight, especially preferably 3 to 7% by weight, based on the finished composition.

Preferred amphoteric surfactants are: N—(C₁₂-C₁₈-alkyl)-β-aminopropionates and N—( C₁₂-C₁₈-alkyl)-β-iminodipropionates as alkali metal and mono-, di- and trialkylammonium salts; N-acylaminoalkyl-N,N-dimethylacetobetaine, preferably N—(C₈-C₁₈-acyl)aminopropyl-N,N-dimethylacetobetaine; C₁₂-C₁₈-alkyldimethylsulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxides, e.g. C₁₂-C₁₈-alkyldimethylamine oxide, fatty acid amidoalkyldimethylamine oxide, alkyl taurates, in particular sodium methylcocoyltaurate (Hostapon® CT, Clariant GmbH), sodium methyllauroyltaurate, isethionates, for example sodium cocoylisethionate.

The weight fraction of the amphoteric surfactants is preferably 0.5 to 20% by weight, particularly preferably 1 to 10% by weight, based on the finished composition.

Furthermore, foam-boosting cosurfactants from the group consisting of alkylbetaines, alkylamidobetaines, aminopropionates, aminoglycinates, imidazoliniumbetaines and sulfobetaines, amine oxides and fatty acid alkanolamides or polyhydroxyamides can be used in the compositions according to the invention.

Preferred surfactants in the compositions according to the invention are alkylbetaines, in particular cocoamidopropylbetaine, amphoacetates, acylglutamates, in particular sodium cocoylglutamate, alkyl ether sulfosuccinates, in particular disodium laurethsulfosuccinate, coconut fatty acid diethanolamide, sodium cocoylisethionate, sodium methylcocoyltaurate and sodium methyllauroyltaurate.

The total amount of surfactants used in the compositions according to the invention is preferably 1 to 70% by weight, particularly preferably 10 to 40% by weight, especially preferably 12 to 35% by weight, based on the finished composition.

Compositions according to the invention in the form of emulsions comprise one or more emulsifiers. These emulsifiers can be chosen from the group of nonionic, anionic, cationic or amphoteric emulsifiers.

Suitable nonionogenic coemulsifiers are preferably addition products of from 0 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan or sorbitol esters; (C₁₂-C₁₈)-fatty acid monoesters and diesters of addition products of from 0 to 30 mol of ethylene oxide onto glycerol; glycerol monoesters and diesters and sorbitan monoesters and diesters of saturated and unsaturated fatty acids having 6 to 22 carbon atoms and optionally ethylene oxide addition products thereof; addition products of from 15 to 60 mol of ethylene oxide onto castor oil and/or hydrogenated castor oil; polyol and, in particular polyglycerol, esters, such as, for example, polyglycerol polyricinoleate and polyglycerol poly-12-hydroxystearate. Likewise preferably suitable are ethoxylated fatty amines, fatty acid amides, fatty acid alkanolamides and mixtures of compounds of two or more of these classes of substance.

Suitable ionogenic coemulsifiers are, for example, anionic emulsifiers, such as mono-, di- or triphosphoric esters, soaps (e.g. sodium stearate), fatty alcoholate sulfates, but in particular cationic emulsifiers, such as mono-, di- and trialkyl quats and polymeric derivatives thereof.

Available amphoteric emulsifiers are preferably alkylaminoalkylcarboxylic acids, betaines, sulfobetaines and imidazoline derivatives.

It is also possible to use naturally occurring emulsifiers, of these preference being given to beeswax, wool wax, lecithin and sterols.

Fatty alcohol ethoxylates are preferably chosen from the group of ethoxylated steryl alcohols, cetyl alcohols, cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether, polyethylene glycol(20) cetylstearyl ether, polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

As ethoxylated alkyl ether carboxylic acid or salts thereof it is advantageously possible to use sodium laureth-11-carboxylate.

An advantageous alkyl ether sulfate is sodium laureth-14 sulfate, and an advantageous ethoxylated cholesterol derivative is polyethylene glycol(30) cholesterol ether. Preference is likewise given to polyethylene glycol(25) soyasterol.

Ethoxylated triglycerides which can be used advantageously are polyethylene glycol(60) evening primrose glycerides.

It is also advantageous to choose the polyethylene glycol glycerol fatty acid esters from the group consisting of polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

Among the sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate are particularly suitable.

The weight fraction of the emulsifier or emulsifiers present in the compositions according to the invention is preferably 0.1 to 20% by weight, particularly preferably 0.5 to 15% by weight, especially preferably 1 to 10% by weight, based on the finished composition.

Suitable cationic polymers are preferably the compounds known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, Polyquaternium 37&mineral oil&PPG trideceth (®Salcare SC95), PVP dimethylaminoethyl methacrylate copolymer, guar hydroxypropyltriammonium chlorides, and calcium alginate and ammonium alginate.

Furthermore, the following may preferably be used: cellulose derivatives; cationic starch; copolymers of diallylammonium salts and acrylamides; quaternized vinylpyrrolidone/vinylimidazole polymers; condensation products of polyglycols and amines; quaternized collagen polypeptide; quaternized wheat polypeptide; polyethyleneimines; cationic silicone polymers, such as, for example, aminomethicones; copolymers of adipic acid and dimethylaminohydroxypropyldiethylenetriamine; polyaminopolyamide and cationic chitin derivatives, such as, for example, chitosan.

The weight fraction of cationic polymers in the compositions according to the invention can preferably be in the range from 0.1 to 10% by weight, particularly preferably in the range from 0.2 to 5% by weight, especially preferably in the range from 0.5 to 2.5% by weight.

The desired viscosity of the compositions can be adjusted by adding thickeners. Of suitability are preferably cellulose ethers and other cellulose derivatives (e.g. carboxymethylcellulose, hydroxyethylcellulose), gelatin, starch and starch derivatives, sodium alginates, fatty acid polyethylene glycol esters, agar agar, tragacanth or dextrin derivatives, in particular dextrin esters.

The synthetic polymers used are various materials, preferably polyvinyl alcohols, polyacrylamides, polyvinylamides, polysulfonic acids, in particular copolymers based on ammonium salts of acrylamidoalkylsulfonic acids and cyclic N-vinylcarboxamides or cyclic and linear N-vinylcarboxamides and also hydrophobically modified acrylamidoalkylsulfonic acid copolymers, polyacrylic acid, polyacrylic acid derivatives, polyacrylic esters, polyvinylpyrrolidone, polyvinyl methyl ether, polyethylene oxides, copolymers of maleic anhydride and vinyl methyl ether, and various mixtures and copolymers of the abovementioned compounds, including their various salts and esters. These polymers can, if desired, be crosslinked or uncrosslinked.

Depending on the intended use, preferred film formers are salts of phenylbenzimidazolesulfonic acid, water-soluble polyurethanes, for example C10-polycarbamoylpolyglyceryl esters, polyvinyl alcohol, polyvinylpyrrolidone copolymers, for example vinylpyrrolidone/vinyl acetate copolymer, water-soluble acrylic acid polymers/copolymers or esters or salts thereof, for example partial ester copolymers of acrylic/methacrylic acid and polyethylene glycol ethers of fatty alcohols, such as acrylate/steareth-20 methacrylate copolymer, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, available for example under the trade name Aristoflex® A 60 (Clariant), and polymeric amine oxides, for example representatives available under the trade names Diaformer Z-711, 712, 731, 751.

Preferably suitable antimicrobial active ingredients are cetyltrimethylammonium chloride, cetylpyridinium chloride, benzethonium chloride, diisobutylethoxyethyldimethylbenzylammonium chloride, sodium N-laurylsarcosinate, sodium-N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (Triclosan), phenoxyethanol, 1,5-pentanediol, 1,6-hexanediol, 3,4,4′-trichlorocarbanilide (Triclocarban), diaminoalkylamide, for example L-lysinehexadecylamide, citrate heavy metal salts, salicylates, piroctoses, in particular zinc salts, pyrithiones and heavy metal salts thereof, in particular zinc pyrithione, zinc phenol sulfate, farnesol and combinations of these active substances.

The compositions according to the invention comprise the antimicrobial compositions preferably in amounts up to 50% by weight, particularly preferably in amounts of from 0.01 to 10% by weight, especially preferably in amounts of from 0.1 to 10% by weight.

Advantageous compositions according to the invention comprise one or more antioxidants. Favorable, but nevertheless optional, antioxidants which can be used are all antioxidants which are customary or suitable for cosmetic and/or pharmaceutical applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. (α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol/kg), also (metal) chelating agents (e.g. α-hydroxyfatty acids, palmitic acid, phytic acid, lactoferrin), α-hydroxyacids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg-ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO4), selenium and derivatives thereof (e.g. selenomethionine), stilbenes and derivatives thereof (e.g. stilbene oxide, trans-stilbene oxide), superoxide dismutase and the derivatives (salts, esters, ethers, sugars, nucleotides, nucleosides, peptides and lipids) of these specified substances which are suitable according to the invention.

For the purposes of the present invention, water-soluble antioxidants can be used particularly advantageously.

The antioxidants can protect the hair against oxidative stress. Preferred antioxidants here are vitamin E and derivatives thereof, and vitamin A and derivatives thereof.

The amount of antioxidants (one or more compounds) in the compositions according to the invention is preferably 0.001 to 30% by weight, particularly preferably 0.05 to 20% by weight, in particular 1 to 10% by weight, based on the total weight of the compositions.

If vitamin E and/or derivatives thereof are the antioxidant or the antioxidants, it is advantageous to choose their respective concentrations from the range from 0.001 to 10% by weight, based on the total weight of the compositions.

If vitamin A, or vitamin A derivatives, or carotenes or derivatives thereof are the antioxidant or the antioxidants, it is advantageous to choose their respective concentrations from the range from 0.001 to 10% by weight, based on the total weight of the compositions.

In one particularly preferred embodiment of the invention, the cosmetic or pharmaceutical compositions comprise antioxidants chosen from superoxide dismutase, tocopherol (vitamin E) and ascorbic acid (vitamin C).

Suitable UV filters are preferably 4-aminobenzoic acid; 3-(4′-trimethylammonium)benzylideneboran-2-one methylsulfate; 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo[2.2.1]heptane-1-methanesulfonic acid and its salts; 1-(4-tert-butylphenyl)-3-(4-methoxyphenyl)propane-1,3-dione, 3-(4′-sulfo)benzylidenebornan-2-one and its salts; 2-ethylhexyl 2-cyano-3,3-diphenylacrylate; polymers of N-[2(and 4)-(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)-4-methyl-6-(2-methyl-3-(1,3,3,3-tetramethyl-1-(trimethylsilyloxy)disiloxanyl)propyl)phenol; bis(2-ethylhexyl) 4,4′-[(6-[4-((1,1-dimethylethyl)aminocarbonyl)phenylamino]-1,3,5-triazin-2,4-yl)diimino]bis benzoate; 3-(4′-methylbenzylidene)-D, L-camphor; 3-benzylidenecamphor; 2-ethylhexyl salicylate; 2-ethylhexyl 4-dimethylaminobenzoate; hydroxy-4-methoxybenzophenone-5-sulfonic acid (Sulisobenzonum) and the sodium salt; and/or 4-isopropylbenzyl salicylate.

Pigments/micropigments which may be used are preferably microfine titanium dioxide, mica-titanium oxide, iron oxides, mica-iron oxide, zinc oxide, silicon oxides, ultramarine blue, chromium oxides.

Suitable gelling agents are all surface-active substances which, dissolved in the liquid phase, form a network structure and thus consolidate the liquid phase. Suitable gelling agents are specified, for example, in WO 98/58625.

Preferred gelling agents are metal salts of fatty acids, preferably having 12 to 22 carbon atoms, for example sodium stearate, sodium palmitate, sodium laurate, sodium arachidate, sodium behenate, potassium stearate, potassium palmitate, sodium myristate, aluminum monostearate, hydroxyfatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures thereof.

Preferably, the compositions according to the invention comprise 0.01 to 20% by weight, particularly preferably 0.1 to 10% by weight, especially preferably 1 to 8% by weight and very particularly preferably 3 to 7% by weight, of gelling agents.

Further additives may be silicone compounds, preferably dimethylpolysiloxanes, methylphenylpolysiloxanes, cyclic silicones, and amino-, fatty acid-, alcohol-, polyether-, epoxy-, fluorine- and/or alkyl-modified silicone compounds, for example phenyltrimethicones from Clariant GmbH such as SilCare® 15M30, SilCare® 15M40, SilCare® 15M50, SilCare® 15M60, caprilyltrimethicones such as SilCare® 31M30, SilCare® 31 M40, SilCare®) 31M 50, SilCare® 31 M 60, alkylmethicones such as SilCare® Silicone 41M10, SilCare® Silicone 41 M15, SilCare® Silicone 41 M20, SilCare® Silicone 41 M30, SilCare® 41M40, SilCare® 41M50, SilCare® 41M65, SilCare® 41M70 or SilCare® 41M80, SilCare® 41M90, trimethylsilyl trimethylsiloxylactate, trimethylsilyl trimethylsiloxyglycolate, trimethylsilyl trimethylsiloxysalicylate, retinoxytrimethylsilane, polyalkylarylsiloxanes and polyethersiloxane copolymers and modified polyorganosiloxanes, for example SilCare® Silicone SEA (Clariant GmbH).

The compositions according to the invention can comprise the abovementioned silicone compounds preferably in the amounts by weight from 0.1 to 20% by weight, particularly preferably 0.2 to 15% by weight, especially preferably 0.5 to 10% by weight, based on the finished compositions.

Suitable carrier materials are preferably vegetable oils, natural and hydrogenated oils, waxes, fats, water, alcohols, polyols, glycerol, glycerides, liquid paraffins, liquid fatty alcohols, sterol, polyethylene glycols, cellulose and cellulose derivatives.

Fungicidal active ingredients which may be used are preferably ketoconazole, oxiconazole, terbinafin, bifonazole, butoconazole, cloconazole, clotrimazole, econazole, enilconazole, fenticonazole, isoconazole, miconazole, sulconazole, tioconazole, fluconazole, itraconazole, terconazole and naftifine, Zn pyrethione and octopirox in the amounts by weight of from 0.05 to 5% by weight, preferably 0.1 to 3% by weight, particularly preferably 0.2 to 2% by weight, based on the finished compositions. The compositions according to the invention can advantageously be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances.

The moisturizing substances available are preferably isopropyl palmitate, glycerol and/or sorbitol, which are preferably used in the amounts by weight 0.1 to 50%.

Superfatting agents which may be used are preferably lanolin and lecithin, nonethoxylated and polyethoxylated or acylated lanolin and lecithin derivatives, polyol fatty acid esters, mono-, di- and triglycerides and/or fatty acid alkanolamides.

Suitable preservatives are preferably phenoxyethanol, parabens, pentanediol or sorbic acid. They are preferably used in the amounts by weight of from 0.001 to 5% by weight, particularly preferably from 0.01 to 3% by weight, especially preferably from 0.1 to 2% by weight, based on the finished compositions.

Dyes which can be used are the substances approved and suitable for cosmetic and pharmaceutical purposes.

Fragrance and/or perfume oils which may be used are individual odorant compounds, e.g. the synthetic products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones includes, for example, the ionones, alpha-isomethylionone and methyl cedryl ketone, the alcohols include anethol, citronellol, eugenol, geraniol, linalool, phenylethyl alcohol and terpineol, and the hydrocarbons include primarily the terpenes and balsams. Preference is given to using mixtures of different odorants which together produce a pleasant scent note.

Perfume oils may also comprise natural odorant mixtures, as are accessible from vegetable or animal sources, e.g. pine, citrus, jasmine, lily, rose or ylang-ylang oil. Essential oils of lower volatility, which are mostly used as aroma components, are also suitable as perfume oils, e.g. sage oil, camomile oil, clove oil, melissa oil, mint oil, cinnamon leaf oil, linden blossom oil, juniper berry oil, vetiver oil, olibanum oil, galbanum oil and ladanum oil.

The acids or alkalis used for adjusting the pH are preferably mineral acids, for example HCl, inorganic bases, for example NaOH, KOH and organic acids, preferably citric acid.

The compositions are preferably adjusted to a pH in the range 2 to 10, preferably pH 3 to 8, particularly preferably 4 to 7.

The examples below are intended to illustrate the invention in more detail in a nonlimiting way. The designation “parts” is to be understood as meaning “parts by weight”.

### EXAMPLES

A. Silicone Oils

1. Preparation of the Silane Mixtures (I) and (II)

1.1 Preparation of Glycidyldiethylamine

298.00 parts of diethylamine were combined with 12.25 parts of water.

Then, with stirring, at 20° C., 377.60 parts of epichlorohydrin were added dropwise over the course of 10 hours. The mixture was then further stirred for a further 10 hours at 20° C., and then 506.7 parts of sodium hydroxide solution, aqueous, 30% strength by weight, were added dropwise. After 3 hours (15-20° C.), the stirrer was switched off. An organic phase (501.5 parts) formed which was separated off. It consisted of about 384.0 parts glycidyldiethylamine, 60.0 parts N,N,N′,N′-tetraethyl-1,3-diamino-2-hydroxypropane, 25.0 parts water, 24.5 parts N,N-diethyl-2-hydroxy-3-chloropropanamine, 1.0 part sodium chloride and 7.0 parts 3-dimethylamino-2-hydroxy-1-propanol.

1.2 Preparation of Silane Mixture (I)

309.00 parts of 3-(2-aminoethylamino)-propyldimethoxymethylsilane were mixed with 505.40 parts of freshly prepared organic phase from 1.1 with stirring and heated to 60° C. A slightly exothermic reaction took place. After about 2 hours, the exothermic reaction subsided, and the mixture was left to react further at 60° C. for 4 hours. It was then cooled to room temperature. Glycidyl groups could no longer be titrated. This was because alkylation of the primary amino group had taken place. 814.4 parts of a silane mixture (I) were thus obtained with the following main components:

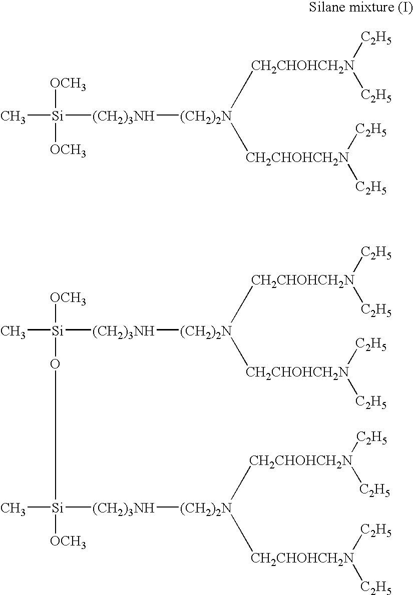

1.3 Preparation of Silane Mixture (II)

286.50 parts of 3-aminopropyldiethoxymethylsilane were mixed with 505.40 parts of the freshly prepared organic phase from 1.1 with stirring at room temperature and heated to 60° C. An exothermic reaction took place, during which the temperature was kept at 60° C. by cooling. As soon as the exothermic reaction had passed, the mixture was left to react further for 4 hours at 60° C. and only then cooled to room temperature. Glycidyl groups could no longer be titrated. This was because alkylation of the primary amino groups of the silane had taken place. This thus gave 791.9 parts of a silane mixture (II) with the following main components:

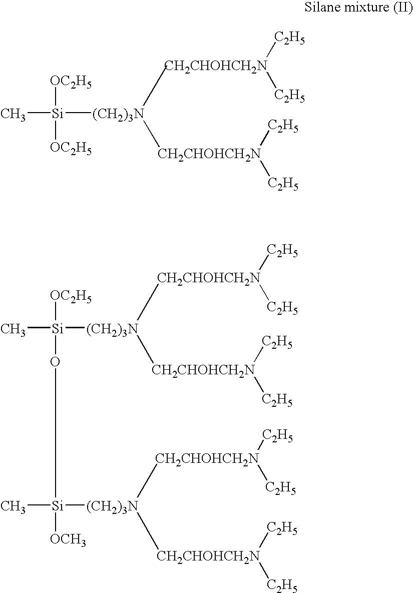

2. Preparation of Silanes (III) and (IV)

2.1 Preparation of Glycidyldipropylamine

404.0 parts of dipropylamine were combined with 12.0 parts of water and cooled to a temperature of 20° C. Then, over the course of 60 minutes, 370.0 parts of epichlorohydrin were added dropwise, during which the temperature was kept between 18 and 20° C. After an after-stirring time of about 20 hours at 20° C., 673.4 parts of aqueous sodium methoxide-methanol solution, 30% strength, were then added dropwise over 60 minutes. A sodium chloride precipitate formed immediately through the formation of the glycidyl compound. Following removal of the sodium chloride by filtration, firstly methanol and then the formed glycidyldipropylamine were distilled off. Between 65 and 80° C. at 8 to 14 mbar, 470 g of glycidyldipropylamine with an equivalent weight of 161.8 (97%) were obtained (yield: 72.5%).

2.2 Preparation of Silane (III)

309.00 parts of 3-(2-aminoethylamino)propyldimethoxymethylsilane were prepared exactly as described under 1.2 using 485.4 parts of glycidyldipropylamine and reacted according to 2.1. 794.4 parts of silane (III) with the following structure were obtained:

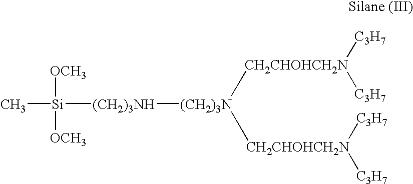

2.3 Preparation of Silane (IV)

286.50 parts of 3-aminopropyldiethoxymethylsilane were prepared exactly as described under 1.2 using 485.4 parts of glycidyldipropylamine and reacted according to 2.1. 771.9 parts of silane (IV) with the following structure were thus obtained:

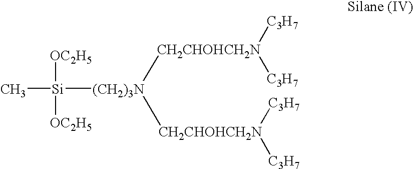

3. Preparation of Silane (V)

187.0 parts of N′-[3-(dimethylamino)propyl]-N,N-dimethylpropane-1,3-diamine were heated to 80° C. 248.0 parts of diethoxy(3-glycidyloxypropyl)methylsilane were then added dropwise, during which the temperature was kept at 80° C. Following the addition of glycidyl, the mixture was left to fully react for a further 4 hours at 130° C. 435.0 parts of silane (V) with the following structure were thus obtained:

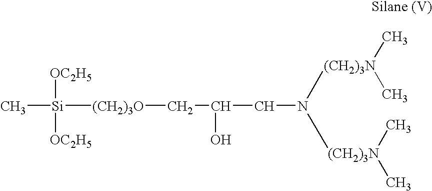

4. Preparation of Polysiloxane (I)

691.0 parts of polydimethylsiloxanediol (viscosity 80 cp=0.08 Pa·s) (polydimethylsiloxanediol L), 28.2 parts of silane mixture (I), and 5.5 parts of a 40% strength solution of benzyltrimethylammonium hydroxide in methanol were mixed together and heated to a temperature of 80° C. with stirring. After 3 hours at 80° C., evacuation to a residual pressure of about 200 mbar was carried out and, at this pressure, the mixture was heated to 150° C. over the course of 60 minutes. Evacuation to a residual pressure of about 50 mbar was then carried out and, after 60 minutes under these conditions under constant residual pressure (50 mbar), the mixture was cooled to room temperature. About 707.0 parts of polysiloxane (I) (viscosity 2660 cp=2.66 Pa·s) and 15.0 parts of distillate were thus obtained.

5. Preparation of Polysiloxane (II)

691.0 parts of polydimethylsiloxanediol (viscosity 80 cp=0.08 Pa·s) (polydimethylsiloxanediol L), 38.73 parts of silane mixture (II) and 5.4 parts of a 40% strength solution of benzyltrimethylammonium hydroxide in methanol were heated to 80° C. with stirring. After 3 hours at 80° C., evacuation to a residual pressure of about 200 mbar was carried out and the mixture was heated to 150° C. at this residual pressure (within about 60 minutes). Evacuation to a residual pressure of 50 mbar was then carried out, and the mixture is distilled at this pressure and at 150° C. for 60 minutes. About 15.8 parts of distillate were thus obtained. After cooling to room temperature (under reduced pressure), about 715.4 parts of polysiloxane (II) (viscosity 900 cp=0.9 Pa·s) were obtained.

6. Preparation of Polysiloxane (III)

691.0 parts of polydimethylsiloxanediol, 55.1 parts of silane (III) and 3.2 parts of a 40% strength solution of benzyltrimethylammonium hydroxide in methanol were heated to 80° C. in a sealed vessel. After 4 hours at 80° C., the pressure reactor was provided with a distillation bridge and evacuated to a residual pressure of 200 mbar. As soon as this pressure was reached, the mixture was heated to 150° C. over the course of 60 minutes. The residual pressure was then reduced to 50 mbar and further stirred for 1 hour at 150° C. Then, at a residual pressure of 50 mbar, the mixture was cooled to room temperature. About 728.0 parts of polysiloxane (III) with a viscosity of 2150 cp=2.15 Pa·s were thus obtained.

7. Preparation of Polysiloxane (IV)

691.0 parts of polydimethylsiloxanediol L, 38.0 parts of silane (IV) and 0.7 parts of a 40% strength solution of benzyltrimethylammonium hydroxide in methanol were heated to 80° C. and left to react at this temperature over a period of 3 hours. Evacuation to a residual pressure of 900 mbar was then carried out and, at this pressure, the mixture was heated to 150° C. over the course of 60 minutes. Evacuation was then carried out until a residual pressure of 50 mbar was achieved and the temperature was kept at 150° C. for 30 minutes. The mixture was then cooled to room temperature and brought to atmospheric pressure using nitrogen. 694.1 parts of polysiloxane (IV) with a viscosity of 1760 cp=1.76 Pa·s were obtained.

8. Preparation of Polysiloxane (V)

The procedure was as for polysiloxane (IV), except 32.1 parts of silane (V) were used instead of 38.0 parts of silane (IV). 696.2 parts of polysiloxane (V) with a viscosity of 1200 cp=1.2 Pa·s were thus obtained.

9. Preparation of Polysiloxane (VI)

419.3 parts of octamethylcyclotetrasiloxane (D4) and 25.3 parts of 1,1,3,3-tetramethyldisiloxane were heated together with 0.43 parts of trifluoromethanesulfonic acid to 80° C. After 4 hours at 80° C., 0.43 parts of magnesium oxide were added, evacuation to a residual pressure of 50 mbar was carried out, and the mixture was heated to 150° C. under these conditions. After 30 minutes at 150° C. and 50 mbar, the mixture was cooled to room temperature under reduced pressure and emptied out over a paper filter. 405.7 parts of a H-terminated polydimethylsiloxane were thus obtained. This product was then heated again to 80° C. under nitrogen. As soon as this temperature was reached, 35 ml of a 3% strength (based on platinum) platinum cyclovinylmethylsiloxane complex (in cyclic methylvinylsiloxanes) (hydrosilylation catalyst) were added, and 42.6 parts of allyl glycidyl ether were added dropwise over the course of about 60 minutes. As soon as the Si—H groups had fully reacted (if not, some more catalyst was to be added), the mixture was heated to 100° C., evacuated to a residual pressure of 50 mbar and kept at 100° C. for 60 minutes. The mixture was then cooled to room temperature. 443.0 parts of glycidyl-terminated polydimethylsiloxane with an equivalent weight of 1334 (equivalent weight of a glycidyl group) were thus obtained. 62.1 parts of N,N,N′,N′-tetramethyldipropylenetriamine were then added and the mixture was heated to 130° C. As soon as the glycidyl groups were no longer tritratable, the mixture was cooled to room temperature. 505.1 parts of polysiloxane (VI) with the following general formula were thus obtained: 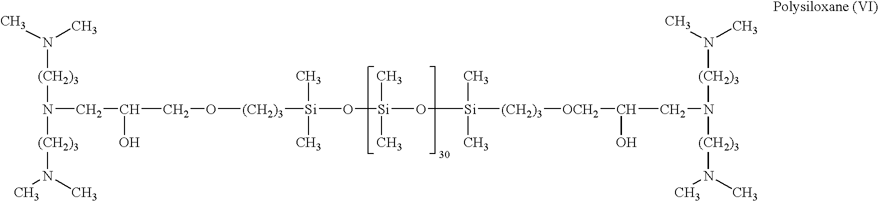
B. End Products

1. Preparation of Quaternary Polysiloxane (I)

200.0 parts of polysiloxane (I) were emulsified with 50.0 parts of tridecanol poly-6,5-ethylene glycol (emulsifier I) and 50.0 parts of water and heated to 40° C. As soon as this temperature was reached, 10.04 parts of dimethyl sulfate were added dropwise. After 6 hours at 40° C., firstly two lots of 200 parts of water and then 40 parts of hexylene glycol were added. A further 70 parts of emulsifier (I) and 180 parts of water were then added. 1000.0 parts of a 20% strength microemulsion of the completely quaternary polysiloxane (I) are thus obtained (quat. polysiloxane I).

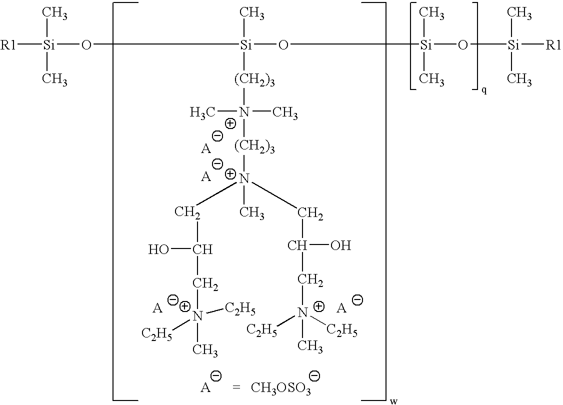

Quat. polysiloxane I

R¹=OH, OMe

w:q˜1:40

2. Preparation of Quaternary Polysiloxane (Ia)

The procedure was as for the preparation of quat. polysiloxane I, but instead of 10.04 parts of dimethyl sulfate, only 6.02 parts were added and before heating to 40° C., following the addition of 50 parts of emulsifier (I), 52.0 parts of water instead of 50 parts were used and also 2.0 parts of dimethyl dicarbonate were added. As soon as the evolution of CO2 had taken place, the mixture was heated to 40° C. and the procedure continued. 1000.0 parts of a 20% strength microemulsion of a quat. polysiloxane Ia were thus obtained with the following functional groups:

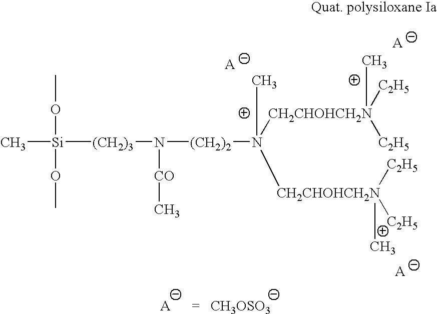

3. Preparation of Quaternary Polysiloxane (II)

200.0 parts of polysiloxane (II) were mixed with 40.0 parts of hexylene glycol and heated to 40° C. 8.86 parts of dimethyl sulfate were then added dropwise and left to fully react for 6 hours at 40° C. Then, 115.0 parts of emulsifier (I) and—as soon as a homogeneous mixture was present—390.0 parts of water at 60° C. were added. A microemulsion formed which was cooled to room temperature by adding 247.0 parts of water and through external cooling. About 1000 parts of microemulsion (quat. polysiloxane II) were thus obtained. The quat. polysiloxane II comprises the following functional groups:

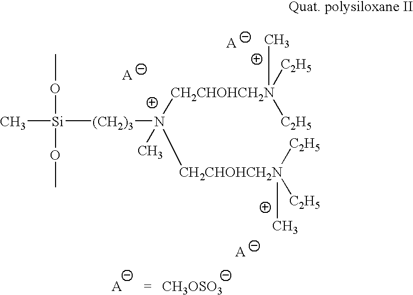

4. Preparation of Quaternary Polysiloxane (III)

The procedure was as for quat polysiloxane II, but with the following amounts and starting materials:

| | | |
|---|---|---|
| Polysiloxane (III) | 200.0 | parts |
| Water (1) | 52.0 | parts |
| Emulsifier (I) (1) | 50.0 | parts |
| Dimethyl dicarbonate | 3.8 | parts |
| Dimethyl sulfate | 10.8 | parts |
| Water (2) | 400.0 | parts |
| Hexylene glycol | 40 | parts |
| Emulsifier (I) (2) | 70 | parts |
| Water (3) | 174 | parts |

About 1000.0 parts of quat polysiloxane III were thus obtained. The emulsified quat polysiloxane III had the following functional groups:

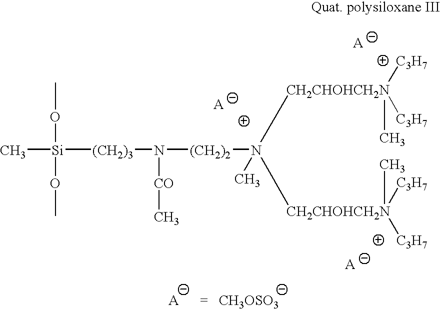

5. Preparation of Quaternary Polysiloxane (IV)

The procedure was as for quat. polysiloxane III but with the following amounts and starting materials:

| | |
|---|---|
| Polysiloxane (IV) | 200.0 parts |
| Hexylene glycol | 40.0 parts |
| Dimethyl sulfate | 8.1 parts |
| Emulsifier (I) | 115.0 parts |
| Water (1) | 390.0 parts |
| Water (2) | 247.0 parts |

About 1000.0 parts of quat. polysiloxane IV were thus obtained. The emulsified quat. polysiloxane IV had the following functional groups:

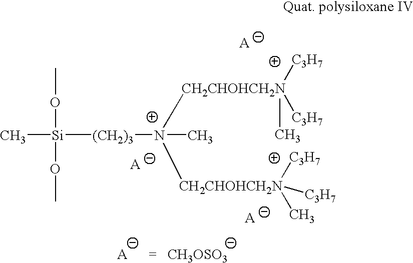

6. Preparation of Quaternary Polysiloxane (V)

The procedure was as for quat. polysiloxane IV, but using polysiloxane (V) instead of (IV). About 1000.0 g of quat. polysiloxane V were obtained. The emulsified quat. polysiloxane V had the following functional groups:

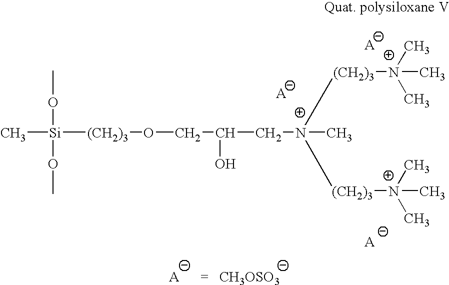

7. Preparation of Quaternary Polysiloxane (VI)

200.0 parts of polysiloxane (VI) were mixed with 100.0 parts of hexylene glycol and reacted at 40° C. with 49.7 parts of dimethyl sulfate for 4 hours. After adding 751 parts of water, 1000.0 parts of end product quat. polysiloxane VI were obtained. The self-dispersed quat. polysiloxane VI had the following structure:

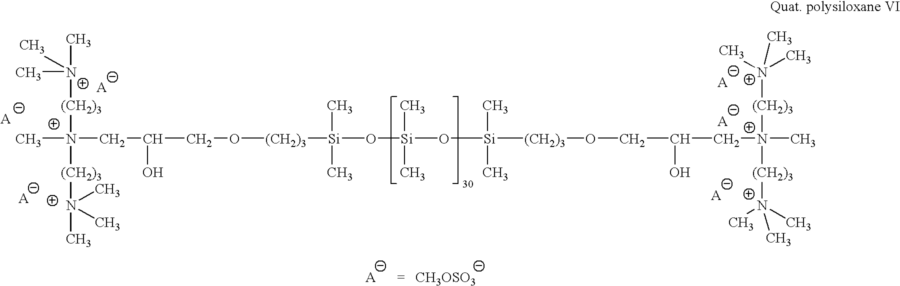

### Example 1

### Color Protection and Shine

Standardized, blond-bleached hair tresses were colored using a standard commercial permanent hair color (Viva Purered, fiery red) under standard conditions. Then, tress A was washed with lauryl ether sulfate:cocoamidopropylbetaine (3:1, 12% by weight active content, H2O ad 100% by weight), and tress B was washed with lauryl ether sulfate:cocoamidopropylbetaine (3:1, 12% by weight active content, H2O ad 100% Al)+quat polysiloxane I (1% by weight active content) 4 times in each case. In a panel of 10 people, tress A is assessed as standard (0). An improvement compared with the standard was evaluated with + (=good), ++ (=very good) or +++ (=exceptional), a deterioration was graded with − or −−.

TABLE 1
Application example color protection and shine
The parameters color intensity, color brilliance, shine, feel and
electrostatic charging after the four washing operations are listed below
(average from all test persons).

| | | Color | | | Electrostatic |
|---|---|---|---|---|---|
| | Color intensity | brilliance | Shine | Feel | charging |
| Tress A | 0 | 0 | 0 | 0 | 0 |
| (standard) | | | | | |
| Tress B | + | ++ | +++ | + | 0 |

Result:

The hair tresses treated with quat. polysiloxane I had significantly lower bleeding of the hair color in the visual and sensory test (increased color intensity, higher color brilliance) and additionally displayed significantly improved shine and a better feel.

### Example 2

### Combability of Hair

Blond, bleached hair tresses were treated with a 2% strength (active) aqueous solution of quat. polysiloxane I and tested with regard to wet and dry combability by a test panel of 10 people (tress B). Genamin CTAC (INCI: Cetrimonium Chloride) serves here as standard (tress A).

The assessment is from −2 (much worse) via 0 (similar to standard) up to +2 (very much better) in unit steps.

The results in Table 2 are average values.

TABLE 2
Combability of hair

| | Wet | Dry |
|---|---|---|
| Tress A (standard) | 0 | 0 |
| Tress B | +1.5 | −1.5 |

The results show that quat. polysiloxane I significantly increases the wet combability compared with the standard. The somewhat poorer values in the case of the dry combability can be compensated for by adding other additives, such as, for example, SilCare Silicone SEA.

The applications below are intended to illustrate the invention in more detail without, however, limiting it thereto (all of the percentages given are percentages by weight).

### Example 3

### Cream Rinse

| | | | |
|---|---|---|---|
| A | Hostacerin ® DGI | Clariant | 1.50% |
| | Cetyl alcohol | | 3.00% |
| B | Genamin ® CTAC | Clariant | 3.30% |
| | Water | | ad 100% |
| | Preservative | | q.s. |
| C | Fragrance | | 0.30% |
| | Quat. polysiloxane I | Clariant | 1.25 |

Preparation:
I Melting of A at about 75° C.
II Heating of B to about 75° C.
III Addition of II to I with stirring and further stirring to 30° C.
IV Addition of C to III at 30° C.
V Adjustment to pH 4.0 with citric acid

### Example 4

### Cream Rinse

| | | | |
|---|---|---|---|
| A | Hostacerin ® DGI | Clariant | 1.50% |
| | Cetyl alcohol | | 4.00% |
| B | Genamin ® CTAC | Clariant | 3.30% |
| | Water | | ad 100% |
| | Preservative | | q.s. |
| C | Fragrance | | 0.30% |
| | Quat. polysiloxane I | Clariant | 0.75 |
| | SilCare ® Silicone SEA | (Clariant) | 0.75 |

Preparation:
I Melting of A at about 75° C.
II Heating of B to about 75° C.
III Addition of II to I with stirring and further stirring to 30° C.
IV Addition of C to III at 30° C.
V Adjustment to pH 4.0 with citric acid

### Example 5

### Cream Rinse with Pearlescent Effect

| | | | |
|---|---|---|---|
| A | Hostacerin ® DGI | Clariant | 1.50% |
| | Cetyl alcohol | | 4.00% |
| B | Genamin ® BTLF | Clariant | 3.30% |
| | Water | | ad 100% |
| | Preservative | | q.s. |
| C | Fragrance | | 0.30% |
| | Quat. polysiloxane I | Clariant | 1.25 |
| D | Citric acid | | q.s. |

Preparation:
I Melting of A at about 75° C.
II Heating of B to about 75° C.
III Addition of II to I with stirring and further stirring to 30° C.
IV Addition of C to III at 30° C.
V Adjustment to pH 4.0 with citric acid

### Examples 6 to 13

### Hair Shampoo

| | Ingredients | 6 | 7 | 8 | 9 | 10 | 11 | 12 | 13 |
|---|---|---|---|---|---|---|---|---|---|
| A | Genapol LRO liq | 39 | 39 | 39 | 39 | 39 | 39 | 39 | 39 |
| | Genagen CAB 818 | 15 | 15 | 15 | 15 | 15 | 15 | 15 | 15 |
| | Aqua dist. | ad 100 | ad 100 | ad 100 | ad 100 | ad 100 | ad 100 | ad 100 | ad 100 |
| B | Genapol L3 | 1 | 1 | 1.25 | 1 | 1 | 1 | 1 | 1 |
| | Quat. polysiloxane I | 1.25 | 1.25 | 1.25 | 1.25 | 1.25 | 1.25 | 1.25 | 1.25 |
| | NaCl | 2.5 | 2.5 | 2.5 | 2.5 | 2.5 | 2.5 | 2.5 | 2.5 |
| | Hydrolyzed silk | | 1 | | | | | | |
| | Guar | | | 1 | | | | | |
| | Octopirox | | | | 0.3 | | | | |
| | SilCare Silicone SEA | | | | | 0.5 | | | |
| | Genapol PGL | | | | | | 1 | | 1 |
| | Eusolex 232 | | | | | | | | 0.1 |
| | Uvinol P25 | | | | | 0.1 | | | |
| | Uvinol MS 40 | | | 0.1 | | | | | |
| | Dyes, fragrances | q.s | q.s | q.s | q.s | q.s | q.s | q.s | q.s. |
| | Citric acid or NaOH | q.s. | q.s. | q.s. | q.s. | q.s. | q.s. | q.s. | q.s. |
| C | NaCl | 2.5 | 2.5 | 2.5 | 2.5 | 2.5 | 2.5 | 2.5 | 2.5 |

Preparation:
I Mixing of components A
II Successive addition of components B to I
III Adjustment of the pH with citric acid or NaOH
IV Adjustment of the viscosity with NaCl

### Example 14

### Shampoo with Color Protection for Colored Hair

| | | | |
|---|---|---|---|
| A | Glucamat DOE-120 | | 2.00% |
| | Emulsogen ® HCO 040 | Clariant | 2.00% |
| B | Water | | ad 100% |
| C | Genapol ® LRO liquid | Clariant | 22.22% |
| | Genagen ® KB | Clariant | 13.33% |
| | Genamin ® KSL | Clariant | 3.33% |
| | Aristoflex ® PEA 70 | Clariant | 2.86% |
| | Sandopan ® DTC, acid | Clariant | 2.20% |
| | NIGAGUARD ® DCB | Clariant | 0.10% |
| | Quat. polysiloxane I | Clariant | 0.50% |
| | Dye | | q.s. |
| | Fragrance | | 0.20% |
| D | NaOH | | |

Preparation:
I Stirring of components A into B and heating to about 60° C. and with stirring, cooling to room temperature
II Successive stirring of components C into I
III Stirring until the formulation appears clear
IV Adjustment to pH 5.5 with D

### Example 15

### Tinting Shampoo

| | | | |
|---|---|---|---|
| A | Genagen ® KB | Clariant | 7.00% |
| | Velsol semipermanent dye | Clariant | 0.50% |
| B | Genapol ® T 500 p | Clariant | 0.50% |
| | Water | | ad 100% |
| C | Genapol ® LRO liquid | Clariant | 30.00% |
| | Genagen ® LAA | Clariant | 3.00% |
| | Genamin ® CTAC | Clariant | 1.00% |
| | Quat. polysiloxane I | Clariant | 0.50% |
| | Tetrasodium EDTA | | 0.10% |
| | NIGAGUARD ® DMDMH | Clariant | 0.30% |
| | Genapol ® PDB | Clariant | 3.00% |
| | Potassium phosphate | | 1.50% |
| D | Citric acid | | |

Preparation:
I Dissolution of the components with stirring
II Mixing of components B and heating until the solution is clear
III Cooling of B to about 35° C. and successive addition of components C to II
IV Stirring of I into III
V Adjustment to pH 5.5 with D

### Example 16

### Hair Gel

| | | | |
|---|---|---|---|
| A | Aristoflex ® AVC | Clariant | 1.40% |
| | Water | | ad 100% |
| B | Diaformer Z-751 | | 3.00% |
| | Alcohol denat. | | 30.00% |
| | Genapol ® C100 | Clariant | 0.40% |
| | Fragrance | | 0.20% |
| C | Dye | | q.s. |
| | Phenonip ® | Clariant | 0.50% |
| D | Quat. polysiloxane I | Clariant | 0.50% |

Preparation:
I Dissolution of components A
II Mixing of components B
III Addition of II to I with stirring
IV Addition of C to III
V Addition of D to IV

### Example 17

### Hair Ends Care

| | | | |
|---|---|---|---|
| A | Water | | 50.0% |
| B | Tylose ® H 100000 G4 | | 1.00% |
| C | Water | | ad 100% |
| D | Genamin ® PDAC | Clariant | 2.50% |
| | Glycerol | | 2.00% |
| | Quat. polysiloxane I | Clariant | 1.00% |
| E | Citric acid | | q.s. |

Preparation:
I Swell B in A
II Successive dissolution of the individual components D in C
III Addition of II to I
IV Adjustment of the pH with E

### Example 18

### Styling Mousse, Clear

| | | | |
|---|---|---|---|
| A | Genapol ® C-100 | (Clariant) | 0.60% |
| B | Water | | ad 100% |
| | Preservative | | q.s. |
| C | Diaformer ® Z-731 | (Clariant) | 6.00% |
| | Glycerol | | 4.00% |
| | Dow Corning 193 Surfactant | | 0.30% |
| | Quat. polysiloxane I | (Clariant) | 1.25% |

Preparation:
I Mixing of A and B
II Successive addition of components C to I

Chemical Name of the Commercial Products Used

| | | |
|---|---|---|
| Aristoflex ® AVC | (Clariant) | Ammonium acryloyldimethyl- |
| | | taurate/NVP copolymer |
| | | (NVP: N-vinylpyrrolidone) |
| Aristoflex ® PEA 70 | (Clariant) | Polypropylene terephthalate |
| Diaformer Z-751 | | Lauryl/stearyl acrylate, |
| | | ethyleneamine oxide, methacrylate |
| | | copolymer |
| Emulsogen ® HCO 040 | (Clariant) | PEG-40 hydrogenated castor oil |
| Eusolex 232 | (Merck) | Phenylbenzimidazolesulfonic acid |
| Genagen ® CAB 818 | (Clariant) | Cocoamidopropylbetaine |
| Genagen ® KB | (Clariant) | Cocobetaine |
| Genagen ® LAA | (Clariant) | Sodium lauroamphoacetate |
| Genamin ® BTLF | Clariant | Behenyltrimonium quat |
| Genamin ® CTAC | (Clariant) | Cetrimonium chloride |
| Genamin ® KSL | (Clariant) | PEG-5 stearylammonium lactate |
| Genamin ® PDAC | (Clariant) | Polyquaternium-6 |
| Genapol ® C100 | (Clariant) | Coceth-10 |
| Genapol ® PDB | (Clariant) | Glycol distearate/laureth-4/ |
| | | cocoamidopropylbetaine |
| Genapol ® LRO liq. | (Clariant) | Sodium laureth sulfate |
| Genapol L3 | (Clariant) | Laureth-3 |
| Genapol PGL | (Clariant) | Glycol distearate/laureth-4/ |
| | | cocoamidopropylbetaine |
| Genapol ® T 500 P | (Clariant) | Ceteareth-50 |
| Guar | | |
| Glucamat DOE-120 | | PEG-120 methylglusose dioleate |
| Hostacerin ® DGI | (Clariant) | Polyglyceryl-2 sesquiisostearate |
| Hydrolyzed silk | | Hydrolyzed amino acid |
| NIPAGUARD DCB | (Clariant) | Phenoxyethanol, methyl- |
| | | dibromoglutaronitrile |
| NIGAGUARD ® | (Clariant) | DMDM hydantoin |
| DMDMH | | |
| Octopirox ® | (Clariant) | Piroctone olamine |
| Phenonip ® | (Clariant) | Phenoxyethanol/methyl-/ethyl-/ |
| | | butyl-/propyl-/isobutylparaben |
| Sandopan ® DTC, acid | (Clariant) | Trideceth-7 carboxylic acid |
| SilCare ® Silicone SEA | (Clariant) | Modified polyorganosiloxane P |
| Tylose ® H 100000 G4 | | Hydroxyethylcellulose |
| Uvinol P 25 | (BASF) | Ethoxylated aminobenzoate |
| Uvinol MS 40 | (BASF) | Diethylhexylbutamidotriazole |

1. A method of making a pharmaceutical or cosmetic composition for treating keratin fibers comprising the step of:
providing a pharmaceutical or cosmetic composition,
adding the keratin fiber to the cosmetic or pharmaceutical composition comprising a polyquaternary polysiloxane of the formula (S1) 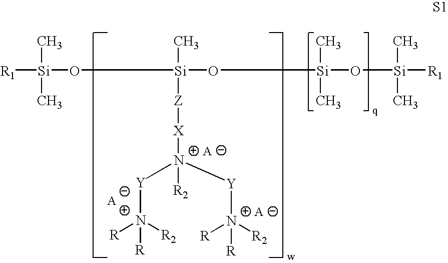
wherein
the sum of (q+w) has a range from 10 to 1500, and the ratio q/w has a range from 5 to 600,
R is C1-C4-alkyl, linear or branched,
R1 is hydrogen, C1-C3-alkyl or C1-C3-alkoxy,
R2 is Cl-C7-alkyl or benzyl,
x is a direct bond, or 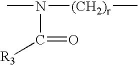
wherein
r is an integer from 1 to 4, and
R3 is C1-C7-alkyl or —NH—C1-C7-alkyl, 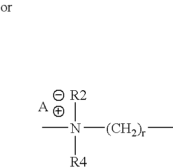
wherein
R2 and r are as defined previously and
R4 is C1-C3-alkyl, 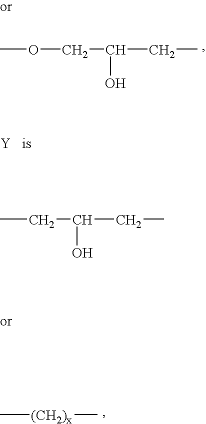
wherein
x is an integer from 1 to 4,
Z is C2-C4-alkylene, linear or branched,
A- is CH3OSO3-, chloride, bromide, iodide or tosylsulfate- or of the formula (S2) 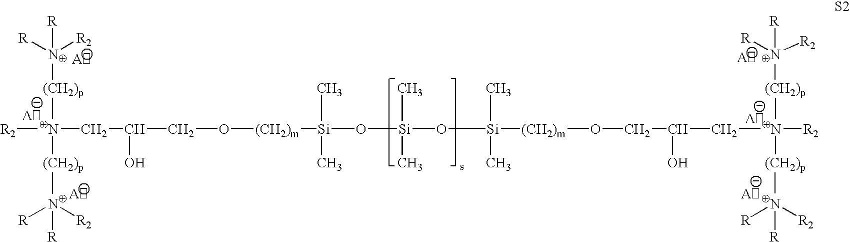
wherein
R, R2 and A- have the same meaning as in formula (S1),
m is an integer from 1 to 4,
p is an integer from 1 to 4, and
s is a number in the range from 5 to 1500.

2. The method according to claim 1, wherein the cosmetic or pharmaceutical composition comprises a polysiloxane selected from the group consisting of: 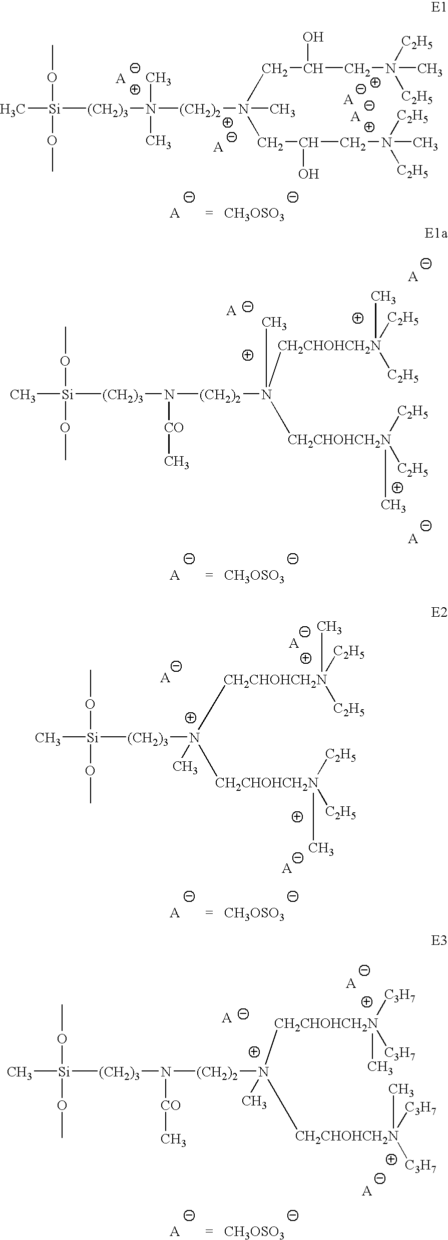 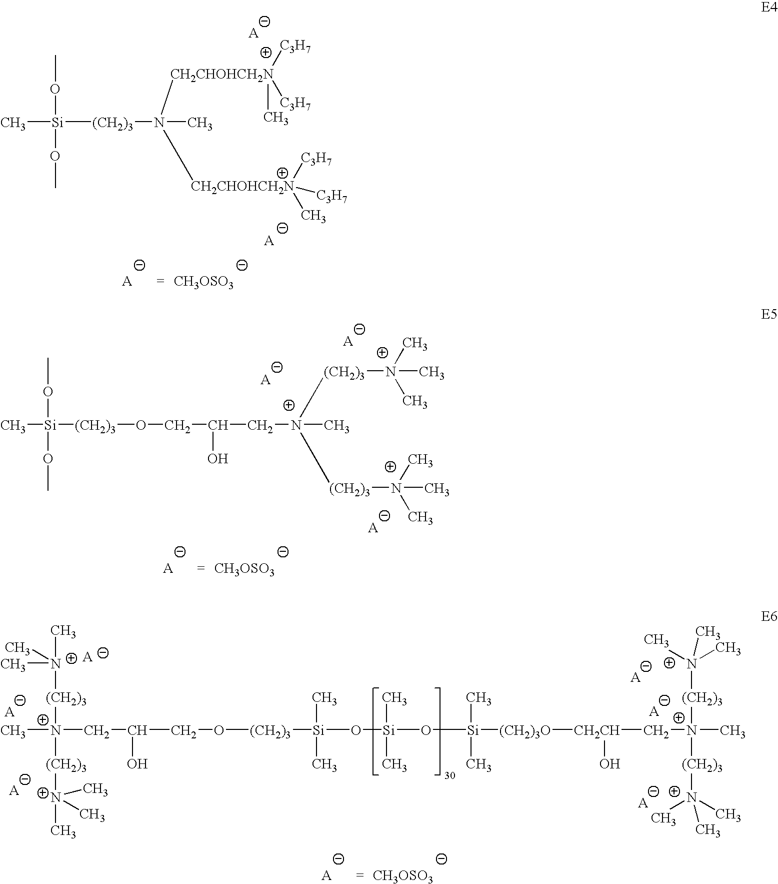
and mixtures thereof.

3. The method according to claim 1, wherein the cosmetic or pharmaceutical composition comprises a polysiloxane of Formula E1 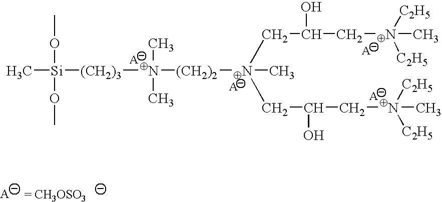
where A- is selected from the group consisting of CH3OSO3-, chloride, bromide, iodide or tosylsulfate- and mixtures thereof.

4. The method according to claim 1, wherein the cosmetic or pharmaceutical composition is in aqueous, aqueous-alcoholic, alcoholic or aqueous-surface-active form, or is based on oil, or is in emulsion, suspension or dispersion form.

5. The method according to claim 1, wherein the cosmetic or pharmaceutical composition is a surfactant-free composition, surfactant-free emulsion, gel, spray, spray foam, mousse or fluid.

6. The method according to claim 1, wherein the cosmetic or pharmaceutical composition is an oil-in-water emulsion with a water fraction of from 5 to 95% by weight.

7. The method according to claim 1, wherein the cosmetic or pharmaceutical composition is an aqueous-alcoholic or alcoholic composition where the alcohols have 1 to 4 carbon atoms, such as ethanol, propanol, isopropanol, n-butanol, isobutanol, t-butanol or glycerol, and alkylene glycols, in particular propylene glycol, butylene glycol or hexylene glycol, or mixtures of said alcohols, or polyethylene glycols with a relative molecular mass below 2000.

8. The method according to claim 1, wherein the cosmetic or pharmaceutical composition are additives for haircare compositions.

9. The method according to claim 1, wherein the polysiloxane is present an amount in the range from 0.01 to 30%, based on the finished cosmetic or pharmaceutical composition.

10. The method according to claim 1 wherein the keratin fiber is human hair.

11. The method according to claim 1 wherein the sum of (q+w) has a range from 15 to 600.

12. The method according to claim 1 wherein the the ratio q/w has a range from 10 to 400.

13. The method according to claim 1 wherein the cosmetic or pharmaceutical composition is in the form of a fluid, foam, spray, gel, mousse, lotion or cream.

14. The method according to claim 6, wherein the cosmetic or pharmaceutical composition is an oil-in-water emulsion with a water fraction of from 15 to 75% by weight.

15. The method according to claim 6, wherein the cosmetic or pharmaceutical composition is an oil-in-water emulsion with a water fraction of from 25 to 85% by weight.

16. The method according to claim 9, wherein the polysiloxane comprises an amount in the range from 0.2 to 10%, based on the finished cosmetic or pharmaceutical compositions.

17. The method according to claim 9, wherein the polysiloxane comprises an amount in the range from 0.5 to 2%, based on the finished cosmetic or pharmaceutical composition.

18. A keratin fiber treatment cosmetic or pharmaceutical composition comprising a polyquaternary polysiloxane of the formula (S1) 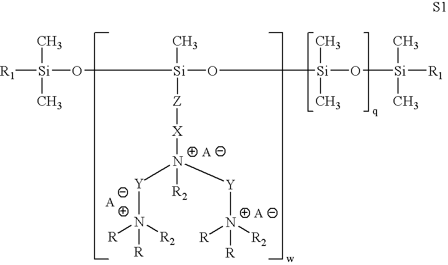
wherein
the sum of (q+w) has a range from 10 to 1500, and the ratio q/w has a range from 5 to 600,
R is C1-C4-alkyl, linear or branched,
R1 is hydrogen, C1-C3-alkyl or C1-C3-alkoxy,
R2 is C1-C7-alkyl or benzyl,
X is a direct bond, or 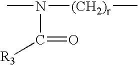
wherein
r is an integer from 1 to 4, and
R3 is C1-C7-alkyl or —NH—C1-C7-alkyl, 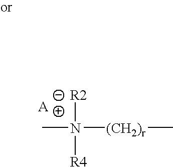
wherein
R2 and r are as defined previously and
R4 is C1-C3-alkyl, 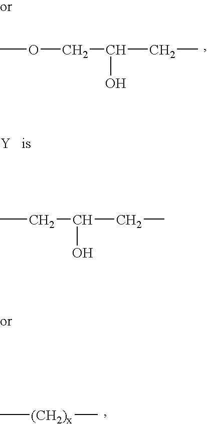
wherein
x is an integer from 1 to 4,
Z is C2-C4-alkylene, linear or branched,
A- is CH3OSO3-, chloride, bromide, iodide or tosylsulfate-
or of the formula (S2) 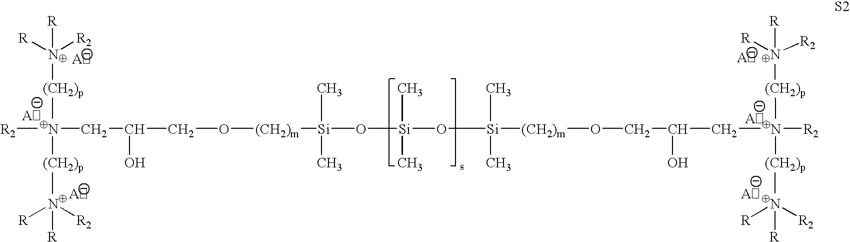
wherein
R, R2 and A- have the same meaning as in formula (S1),
m is an integer from 1 to 4,
p is an integer from 1 to 4, and
s is a number in the range from 5 to 1500.

19. A keratin fiber treated in accordance with claim 1.

20. A method of using a polysiloxane of the formula (S1) 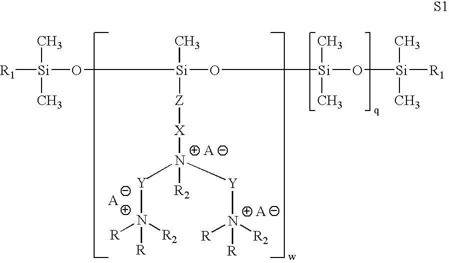
wherein
the sum of (q+w) has a range from 10 to 1500, and the ratio q/w has a range from 5 to 600,
R is C1-C4-alkyl, linear or branched,
R1 is hydrogen, C1-C3-alkyl or C1-C3-alkoxy,
R2 is C1-C7-alkyl or benzyl,
X is a direct bond, 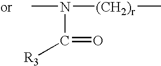
wherein
r is an integer from 1 to 4, and
R3 is C1-C7-alkyl or —NH—C1-C7-alkyl,
or 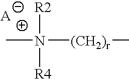
wherein
R2 and r are as defined previously and
R4 is C1-C3-alkyl, 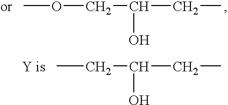
or
—(CH2)x-,
wherein
x is an integer from 1 to 4,
Z is C2-C4-alkylene, linear or branched,
A- is CH3OSO3-, chloride, bromide, iodide or tosylsulfate-
or of the formula (S2) 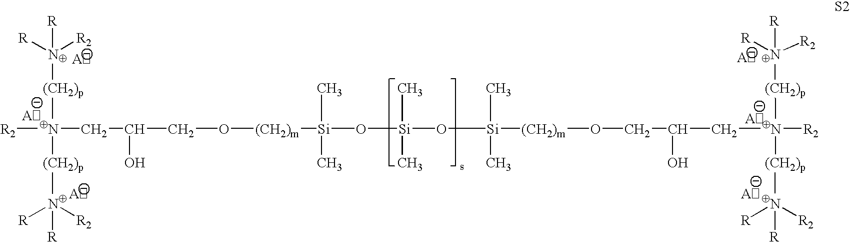
wherein
R, R2 and A- have the same meaning as in formula (S1),
m is an integer from 1 to 4,
p is an integer from 1 to 4, and
s is a number in the range from 5 to 1500
comprising the step of adding the polysiloxane to a cosmetic or pharmaceutical composition in an amount necessary to treat keratin fiber.
